(19) United States
(12) Patent Application Publication
(10) Pub. No.: US 2015/0112699 A1
(43) Pub. Date: Apr. 23, 2015

(54) HEALTH MONITORING METHOD

(71) Applicant: BEESKET GLOBAL INC., Seoul (KR)

(72) Inventor: Seung Hoon Cho, Seoul (KR)

(21) Appl. No.: 14/376,616

(22) PCT Filed: Dec. 24, 2012
(86) PCT No.: PCT/KR2012/011368

§ 371 Date: Aug. 5, 2014

(30) Foreign Application Priority Data
Jun. 12, 2012 (KR) 10-2012-0062385

(51) Int. Cl. A61B 5/00; G06F 19/00

(52) U.S. Cl. CPC A61B 5/486; A61B 5/4866; A61B 5/742; G06F 19/36
USPC 705/2

(57) ABSTRACT

Disclosed is a health monitoring method. The present invention includes: a step of classifying samples by weight and displaying information related to the samples in individual capsules, and a step of reading the information in the individual capsules loaded on a loading module, and loading module information, reading information on a mixed sample in which recipe information on each individual sample and the loading module are mixed with each other using the memory, and analyzing and displaying nutrient composition or caloric content information on the mixed sample. Accordingly, the capsules can be combined in the loading module in a simple manner and the nutrient composition and caloric content of mixed food can be easily determined, thus the optimal nutrient composition or caloric content can be provided according to the health conditions of the user.

### CROSS REFERENCE TO RELATED APPLICATIONS AND CLAIM OF PRIORITY

This patent application is a National Phase application under 35 U.S.C. §371 of International Application No. PCT/KR2012/011368, filed 24 Dec. 2012, which claims priority to Korean Patent Application No. 10-2012-0062385, filed 12 Jun. 2012, entire contents of which are incorporated herein by reference.

### BACKGROUND

1. Field

The present invention relates to a health monitoring method. More particularly, the present invention relates to a health monitoring method in which foods are classified by food name (hereinto referred to as kind) or food quantity by weight (hereinto referred to as weight), and are displayed according to individual nutrients; and in which nutrients of mixtures of foods are specially managed such that the user can consume nutrients in a balanced way.

2. Description of the Related Art

With increases in concern about one's health along with an improvement of quality of life, instruments relevant to diet, dietary habits, and exercise; as well as systems for checking and managing body fat, blood sugar, blood pressure, and other health indices, have recently been widely utilized.

These systems are configured to receive exercise data, food intake, and health indices measured directly by the user; transmit the input data via a communication network to a server wherein the data is managed and stored in the form of a database; and provide the user with information based on their data via written messages, graphs, and vocal responses.

Accordingly, it is inconvenient to utilize these systems because the direct inputting of information, such as exercise and food intake information requires the user to have expert-level knowledge about the information.

In addition, conventional systems suffer from the disadvantage of being insufficient in providing adequate information about nutrients because their structures are adapted to receiving only food intake, are difficult to operate, and are prone to omissions of information because it is impossible to input information, such as food intake, in real time.

For patients who need controlled diets, an assistant who checks foods consumed by patients, or a device that assists in inputting information regarding these foods, may be necessary.

In prepared food venues, deli-counters, or bakeries, the consumer often samples items before buying. Even in this case, individual samples must be assessed for calories or analyzed for information about nutrient composition at, for example, a deli-counter, which lengthens the waiting time at the front of the counter.

Although bar codes are utilized, they are also inconvenient because individual items must either be scanned or their data must be input manually.

### SUMMARY

Accordingly, the present invention has been made with the above issues in mind, wherein in one embodiment, provided is a health monitoring method wherein standardized food capsules are created according to the kind or weight of the food, and loaded on a loading module having information about food recipes; whereby the capsules can be combined to manage nutrient composition or caloric content of mixed foods.

In another embodiment, provided is a health monitoring method wherein foods are readily combined according to the kind or weight of the food in order to provide optimal nutrition or caloric consumption.

In yet another embodiment, provided is a health monitoring method wherein a results report, in which nutrient composition or caloric content of an optimally mixed food are recorded, is managed; not only to maintain certain nutritional guidelines, but also to readily suggest the mixed food again in the future.

In one embodiment, provided is a health monitoring method for maintaining health, using a system including individual capsules further including a first recording medium on which sample information about at least one of: the kind, place of origin, shipment date, weight, price, image, and nutrient composition or calories by weight is recorded; a loading module including one or more receiving cells adapted to accommodate a plurality of the individual capsules, and a second recording medium on which recipe information about different beverages, price information about individual recipes, and images of the beverages are recorded; a memory of a control unit in which the recipe information of the loading module, and information about nutrients or calories taken from mixtures of samples from individual capsules accommodated with the loading module are stored, whereby data of the memory is managed, the health monitoring method including:

(a) classifying samples by weight and kind, and displaying the classified information on the individual capsules;

(b) accommodating at least one selected from among the individual capsules with the loading module;

(c) reading the information displayed on the individual capsules loaded on the loading module, and the recipe information of the loading module; and

(d) reading information on a mixed sample in which the recipe information on each individual sample, and the information of the loading module, both read in step (c), are mixed with each other, using the memory, and analyzing and displaying information on nutrient composition or caloric content of the mixed sample.

In one embodiment of the present invention, the health monitoring method further includes allowing the control unit to make a results report in which the recipe information of the loading module and the information of the individual capsules accommodated with the loading module of step (b) are used as input sample information while the analyzed information of the mixed sample of step (d) is used as a mixture sample information, storing the results report in the memory by identification (I.D.), and outputting the results report, together with the I.D., and (f) allowing the control unit to read the I.D. from the output results report and to extract the information of the mixed sample corresponding to the read I.D. from the memory, thereby preparing a mixed sample identical in nutrient composition or caloric content to the read mixed sample.

According to the health monitoring method of the present invention, the capsules can be combined in the loading module in a simple manner, and the nutrient compositions and caloric contents of the mixed foods can thereby be easily known, and thus the optimal nutrients or calories can be provided according to the health conditions of the user.

In the health monitoring method of the present invention, the capsules may be modified, if necessary, to change the nutrients or calories of the mixed foods, thereby allowing the user who must manage their nutrient or caloric intake to easily monitor his or her health.

Additionally, the results reports on nutrient composition or caloric content of optimally mixed foods are not only administered to manage constant nutrition, but also used to easily provide the optimally mixed foods.

### BRIEF DESCRIPTION OF THE DRAWINGS

FIG. 1 is a block diagram illustrating the construction of a health monitoring system as in one embodiment of the present invention.

FIG. 2 is a flow chart illustrating the health monitoring method as in one embodiment of the present invention.

FIGS. 3 to 6 illustrate the construction of a capsule and a loading module as in one embodiment of the present invention.

FIG. 7 illustrates another embodiment wherein the construction of the capsule and the loading module is altered.

FIG. 8 depicts a display of the information extracted from the capsule and the loading module by the reader, as in one embodiment of the present invention.

FIG. 9 shows an illustrated results report display as in one embodiment of the present invention.

### DETAILED DESCRIPTION

The terminologies and words used herein are not limited to those commonly defined in dictionaries, but should be interpreted as having a meaning that is consistent with their meaning in the context of the relevant art and the present disclosure, and will not be interpreted in an idealized or overly formal sense unless expressly so defined herein.

It will be further understood that the term “include” when used in this specification specifies that an element may further include another element unless the context clearly indicates otherwise. Further, the term, such as unit“, group”, “module”, “device”, etc, used herein means a unit that can have at least one function or operation and may be formed by a combination of hardware or software or both.

Hereinbelow, embodiments of the present invention will be described in detail with reference to the accompanying drawings.

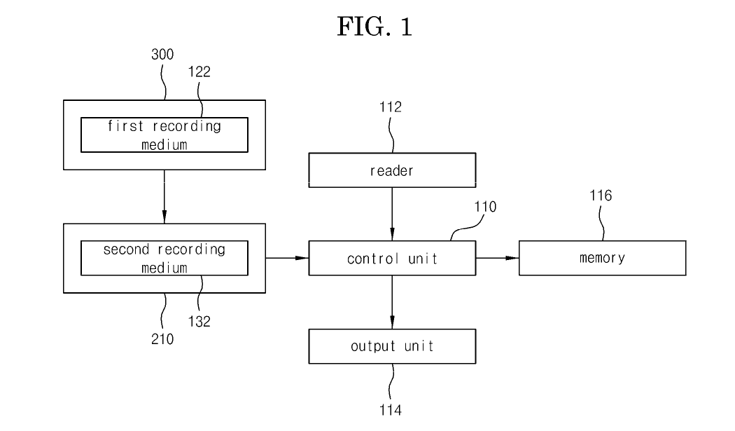

FIG. 1 is a block diagram illustrating the construction of a health monitoring system according to one embodiment of the present invention. As shown in the drawing, the health monitoring system using a health monitoring method of the present invention, may include: a capsule 300 for displaying or storing information of samples; a loading module 210 configured to be loaded with a plurality of capsules 300 and to display or store recipe information; a reader 112 for reading information concerning the capsules 300 and the loading module 210; an output unit 114 for outputting nutrients or calories of individual foods or mixed foods produced by mixing the individual foods; a memory 116 for storing information concerning nutrients or calories of individual samples by weight of the samples, which can be obtained from the information of the capsules and of the loading module 210, and for storing information concerning nutrients or calories of a mixed sample produced by mixing the samples; and a control unit 110 for analyzing both the information concerning the samples of the respective capsules 300 loaded in the loading module 210, and the information concerning nutrients or calories of the mixed sample produced by mixing the samples, which have been read by the reader 112 based on the information concerning both the capsules 300 and the loading module 210, which has been read by the reader 112.

The capsule 300 may include a first recording medium 122 that is configured to display or store information concerning at least one of the kind, place of origin, shipment date, and weight of the individual samples, and the nutrients or calories of the samples by weight.

The loading module 210 is configured as a cylindrical body that defines therein a plurality of receiving cells 218 having respective open tops, so a plurality of capsules 300 can be loaded in the respective receiving cells 218 of the loading module. A holding step 214 is formed in the lower part of each of the receiving cells 218, so a capsule 300 can be stably held in the receiving cell 218.

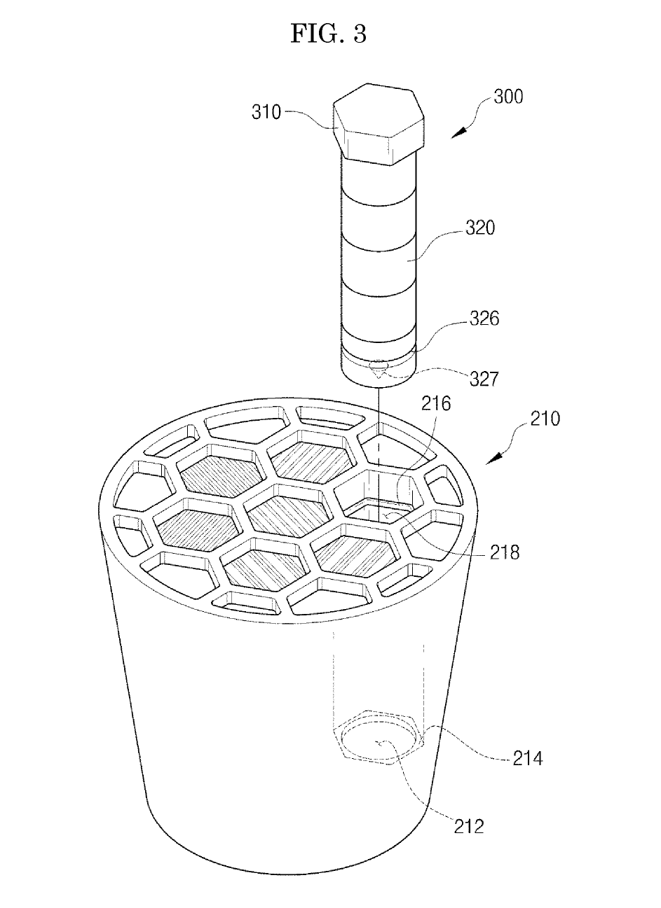

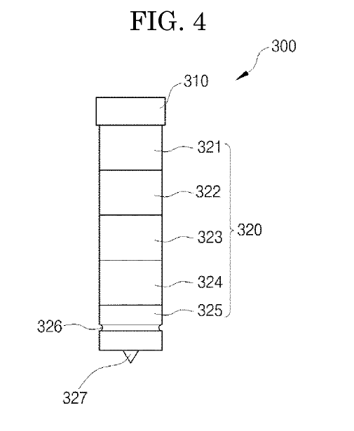

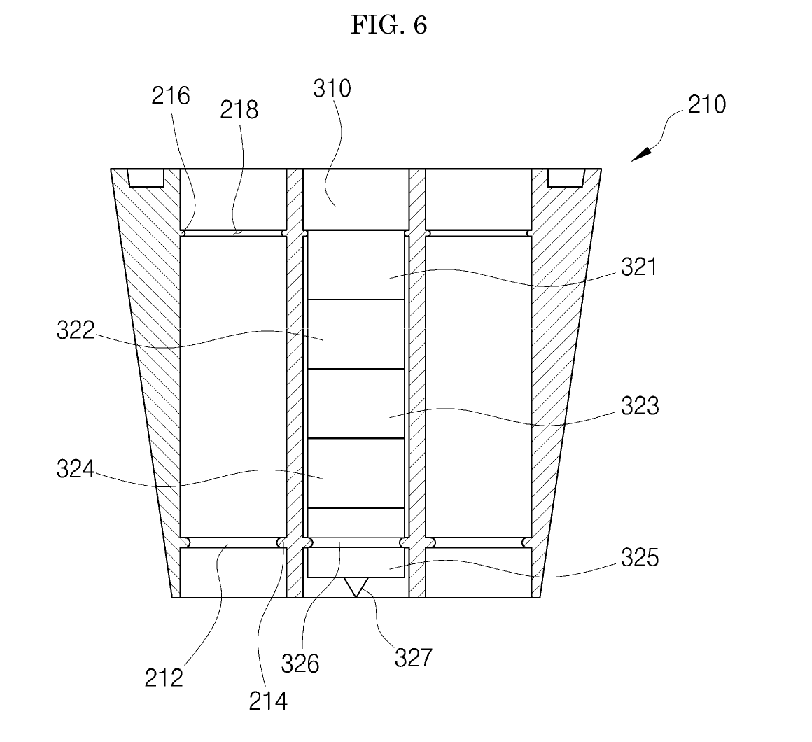

FIGS. 3 to 6 illustrate the construction of a capsule and a loading module as in one embodiment, which may be used in the present invention. As shown in the drawings, the capsule 300 may be provided with (i.e., thereon) or may be embedded with a first recording medium 122 that can display or store information concerning at least one of the kind, place of origin, shipment date, and weight of individual samples, and the nutrients or calories of the samples by weight.

To this end, each of the capsules 300 includes: an upper part having a cylindrical stopper 310; a cylindrical body 320; a groove 326 formed around the lower part of the outer circumferential surface of the cylindrical body 320; and a cone wedge 327 provided on the lower end of the cylindrical body 320.

Here, the groove 326 may be formed as one or more grooves in each of the capsules 300, and the location of the groove 326 may be determined such that the groove 326 can be aligned with the holding step 214. In other words, both the groove 326 and the cone wedge 327 may be appropriately formed as desired.

In the present invention, to shape the capsules 300 and the loading module 210 in the form of a honeycomb, at least one receiving cell 218, having a honeycomb cell shape, may be formed in the loading module 210, and each of the capsules 300 may be formed by combining one or more cylindrical bodies 320 with each other. To this end, each of the cylindrical bodies 320 may be provided with a female locking portion in an upper part thereof, and with a male locking portion in a lower part thereof, so that the male locking portion of one cylindrical body 320 can be combined with the female locking portion of another cylindrical body 320. Here, the cylindrical bodies 320 of each capsule may be configured to have different colors, different images, or different patterns according to the kind or weight of the samples.

Further, each of the capsules 300 is provided with a stopper 310 functioning as a head of the capsule. The capsule also includes one or more cylindrical bodies 321 to 325 (i.e., in sum 320), and a cone wedge 327 protruding downward from the lower end of the lowest cylindrical body 325, thereby realizing an image in which bee-shaped capsules 300 are loaded in the receiving cells of the honeycomb-shaped loading module 210.

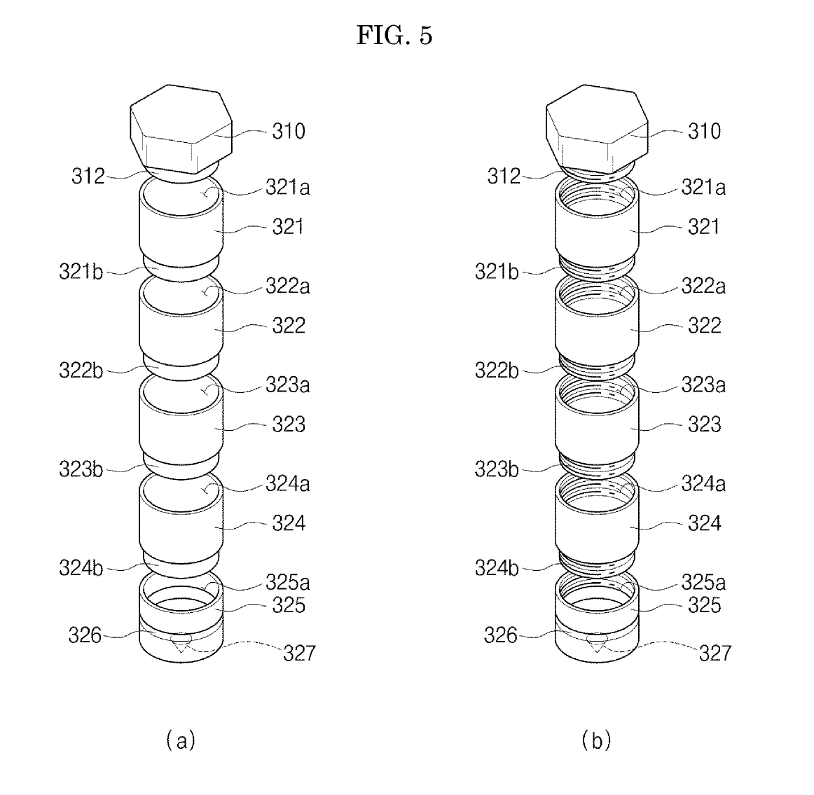

Specifically described with reference to FIG. 5(a), the lower part of the stopper 310 is provided with a male locking portion 312 configured to be inserted into a female locking portion 321a of a first cylindrical body 321, and the lower part of the first cylindrical body 321 is provided with a male locking portion 321b configured to be inserted into a female locking portion 322a formed in the upper part of a second cylindrical body 322. In the same manner, the lower part of the second cylindrical body 322 is provided with a male locking portion 322b configured to be inserted into a female locking portion 323a formed in the upper part of a third cylindrical body 323, the lower part of the third cylindrical body 323 is provided with a male locking portion 323b configured to be inserted into a female locking portion 324a formed in the upper part of a fourth cylindrical body 324, and the lower part of the fourth cylindrical body 324 is provided with a male locking portion 324b configured to be inserted into a female locking portion 325a formed in the upper part of a fifth cylindrical body 325. Further, a groove 326 is formed around the outer circumferential surface of the fifth cylindrical body 325 so as to be removably engaged with the holding step 214 formed in the receiving cell 218, and a cone wedge 327 protrudes downward from the lower end of the fifth cylindrical body 325.

Although the female locking portions and the male locking portions may be configured such that they are combined with each other as described above, the combination of the male and female locking portions may be realized by a screw-type engagement, as shown in FIG. 5(b).

When the capsule 300 having the above-mentioned construction is inserted into a receiving cell 218 of the loading module 210, such that the groove 326 of the capsule 300 is engaged with the holding step 214, the loading of the capsule 300 in the loading module 210 is realized.

In other words, when the stopper 310 of the capsule 300 is held by a holding protrusion 216 of the loading module 210, and the groove 326 of the capsule 300 is engaged with the holding step 214, the capsule 300 can be stably held in the loading module 210 such that the capsule can be prevented from movement or removed undesirably.

In one embodiment, to display colors on the capsules and on the loading module, different colors may be displayed on the stopper 310, the second and fourth cylindrical bodies according to the kind and weight of the samples (e.g., displayed in yellow), and black may be displayed on the first, the third, and the lowest cylindrical bodies, so a bee-like image may be formed.

Referring to the accompanying drawings, the loading module 210 is provided with at least one receiving cell 218 that is configured to vertically receive a capsule 300. The receiving cell 218 may have a hollow cylindrical shape, with the holding protrusion 216 and the holding step 214 formed in the upper and lower parts of the inner circumferential surface of the receiving cell 218, respectively. The receiving cell 218 is provided with a second recording medium recorded with recipe information of different beverages and pricing information of recipes.

The second recording medium may be recorded with nutrient information of recipes of beverages and pricing information of the beverages.

The holding protrusion 216 is configured to hold the cylindrical stopper 310 of a capsule 300 in the receiving cell 218 such that the capsule 300 can be prevented from being undesirably moved downward into the receiving cell 218. When the capsule 300 is inserted into the receiving cell 218, the holding step 214 is removably engaged with the groove 326 of the capsule 300, therefore, the holding step 214 may be at a location corresponding to the location of the groove 326 of the capsule 300 when received in the receiving cell 218.

Further, recipe titles, for example, smoothies, Ade, yogurts, etc., may be displayed on the outer circumferential surface of the loading module, or the images of recipes may be differentially displayed on the outer circumferential surface of the loading module.

For example, the recipe for preparing a smoothie may be displayed in red on the outer circumferential surface of the loading module 210, the recipe for Ade may be displayed in green, and the recipe for yogurt may be displayed in yellow.

In one embodiment, radio frequency identification (RFID) or near field communication (NFC), which can be used for local area radio communication, may be used as the recording medium.

Further, in another embodiment, the construction of the capsule and the loading module of the present invention may be altered without limitation to the above-mentioned construction.

Although the above-described embodiment of the capsule includes four bodies, the capsule may include one, two, or more bodies, as desired. Further, although the above-described embodiment of the loading module has seven receiving cells, the loading module may be configured to have two, three, or more receiving cells in consideration of the number of samples of the capsules loaded into the loading module.

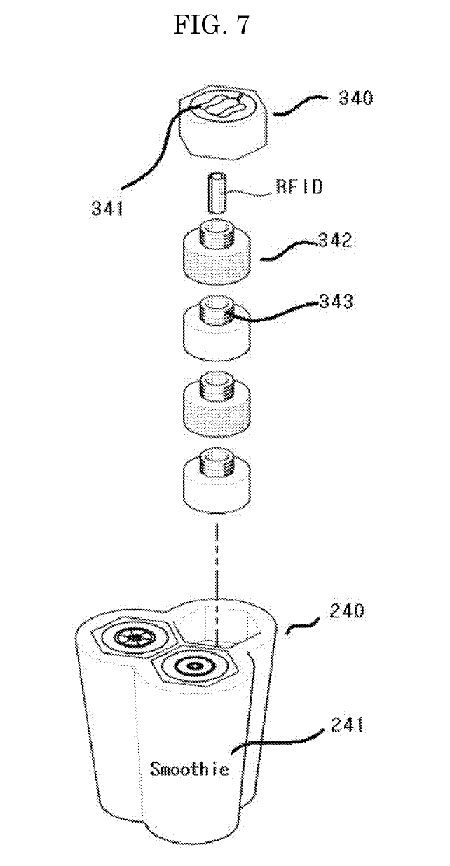

For example, in another embodiment as shown in FIG. 7, the construction of the capsule and the loading module is altered. As shown in the drawing, the loading module 240 includes three receiving cells, with a display part provided on the body of the loading module so as to show a recipe (e.g., a Smoothie). Further, the capsule may be provided with an image 341 on the upper surface of the stopper 340 so as to show the kind of sample, and each of the bodies 342 may be provided at the center thereof with a hollow locking member 343 such that each body 342 can be combined with another body 342, and the hollow locking member 343 may be configured such that an RFID can be installed therein.

The reader 112 is configured to receive the information stored in the first recording medium 122 of each of the capsules 300 or the information stored in the second recording medium 132 of the loading module 210.

Accordingly, when the recording media of both the capsules 300 and the loading module 210 are formed as barcodes, the reader is formed as a barcode reader, and when the recording media are formed as radio frequency (RF) tags, the reader is configured to function as an RF receiver.

The output unit 114 is configured such that it is operated under the control of the control unit 110 and can produce input sample information from both the information of the capsules 300 loaded in the loading module 210 and the recipe information of the loading module 210; can produce mixed sample information from the nutrient information or calorie analyzing information of a mixed sample; and can print resulting reports of the input sample information and the mixed sample information. The resulting reports will be described in detail (infra).

The memory 116 is configured such that it can store the information of recipes and the information of nutrients or calories of individual samples by weight, in which the samples can be mixed with the recipes. Further, the memory is configured to store the information of nutrients or calories that can be obtained from the respective mixed samples after defining the mixed samples by mixing the samples.

In the present invention, different recipes are differently assessed for nutritional information even though they are identical in individual samples.

Data of nutrient composition and caloric content of individual and mixed samples are summarized by recipe in Table 1, below. Among the recipes are: smoothie, yogurt, and Ade.

Individual samples to be mixed for the recipes are listed in the “Kind of Capsule” column, along with their nutrient composition and caloric content.

For example, when 50 g of aloe is used for a smoothie, it is assessed to have 50 kcal, and contain 2.4 mg of carbohydrate, 2.0 mg of dietary fiber, 18 mg of calcium, 38 mg of potassium, 2.0 mg of vitamin A (VA), and 70 mg of vitamin C (VC).

TABLE 1

| | Kind of | | Calorie | Carbo- | | | Dietary | | | | | | | | |
|---|---|---|---|---|---|---|---|---|---|---|---|---|---|---|---|
| Recipe | Capsule | Wt | (Kcal) | hydrate | Protein | Lipid | Fiber | Ca | Fe | K | VA | VB1 | VB2 | VC | VE |
| Smoothie | Aloe | 50.0 | 8.0 | 2.4 | 0.0 | 0.0 | 2.0 | 18.0 | 0.0 | 38.0 | 2.0 | 0.0 | 0.0 | 70.0 | 0.0 |
| | Mulberry | 50.0 | 23.0 | 5.15 | 0.8 | 0.1 | 2.05 | 30.5 | 1.0 | 101.5 | 4.5 | 0.65 | 0.06 | 2.0 | 0.15 |
| | Orange | 50.0 | 21.5 | 5.60 | 0.45 | 0.05 | 0.95 | 16.5 | 0.1 | 63.0 | 7.5 | 0.06 | 0.01 | 4.5 | 0.20 |
| | Cucumber | 50.0 | 5.5 | 1.15 | 0.55 | 0.15 | 0.75 | 15.0 | 0.3 | 156 | 4.5 | 0.02 | 0.01 | 4.5 | 0.2 |
| | Corn | 50.0 | 61.5 | 12.70 | 3.30 | 0.70 | 1.35 | 10.5 | 1.15 | 163 | 3.5 | 0.04 | 0.04 | 0.0 | 0.7 |
| Yogurt | ∘ ∘ ∘ | | | | | | | | | | | | | | |
| Ade | Cranberry | 40.0 | 46.20 | 12.06 | 0.12 | 0.03 | 1.90 | 2.40 | 0.60 | 0.09 | 25.50 | 0.0 | 0.01 | 3.90 | 0.06 |
| | Kiwi | 40.0 | 47.70 | 12.72 | 0.27 | 0.15 | 1.01 | 9.00 | 0.09 | 81.3 | 2.4 | 0.0 | 0.01 | 8.1 | 0.39 |
| | Tomato | 40.0 | 35.70 | 9.39 | 0.27 | 0.03 | 0.74 | 2.7 | 0.09 | 53.4 | 27.0 | 0.01 | 0.0 | 3.3 | 0.18 |
| | Pineapple | 40.0 | 38.40 | 10.29 | 0.12 | 0.03 | 1.0 | 3.0 | 0.12 | 32.1 | 0.0 | 0.03 | 0.0 | 4.5 | 0.0 |
| | Green | 40.0 | 45.60 | 12.18 | 0.09 | 0.06 | 0.79 | 1.5 | 0.09 | 36.0 | 0.6 | 0.02 | 0.0 | 0.6 | 0.15 |
| | grape | | | | | | | | | | | | | | |

Individual capsules and mixed samples thereof by recipe are measured for their nutritional and caloric content in the same manner, and the measurements are graphically represented according to the combination of the individual samples.

In these experiments, the calories of the mixed samples were measured to be slightly higher than the sum of those of the individual samples. As for the individual nutrients, their weights were measured to be slightly higher or lower in the mixed samples than in the individual samples.

Accordingly, the memory 116 may display the recipe information of the loading modules 210, and information on the nutrient composition and caloric content of the individual samples of the individual capsules 300 accommodated with the loading modules 210 by weight, obtained via arithmetic conversion, but may, as described above, store nutrient composition and caloric content of the individual samples (i.e., capsules and loading modules) by weight, classify the number of cases combining individual samples, and calculate and store information on the nutrient composition and caloric content to be taken from the combinations in order to provide more precise data results.

The memory is designed to store the mixed sample information by I.D., so that management of the same I.D. can manage the mixed sample according to the kind of the capsule 300.

The control unit 110 is configured to store the nutrient composition and caloric content of the mixed samples in the memory after the recipe information of the loading module 210, and the samples of individual capsules 300 accommodated with the loading modules 210 are arithmetically converted by weight. A results report may then be generated wherein the recipe information of the loading modules 210, and the sample information of the individual capsules 300 loaded to the loading modules 210 are used as input sample information, while the information on nutrient composition or caloric content of the mixed samples serves as mixed sample information, thereby generating an output of the results report via an output unit 114.

Thus, the control unit 110 may be set at a counter, and operate to read information about the mixtures of samples selected by the user from the memory 116, display the results report on a display device while outputting the report, and store the combinations of the samples in the memory 116, thereby managing the combinations by I.D.

Of course, when the information regarding individual and mixed samples is displayed on a separate display device, the user may modify the information of the individual samples (i.e., individual capsules) whereby the nutrition of the user can be managed more conveniently.

In addition, if the results report output along with the I.D. is used, the I.D. is read so that the information on the mixed sample corresponding to the I.D. can be retrieved from the memory 116, and can be utilized to prepare a mixed sample identical in nutrient composition or caloric content to the reference mixed sample.

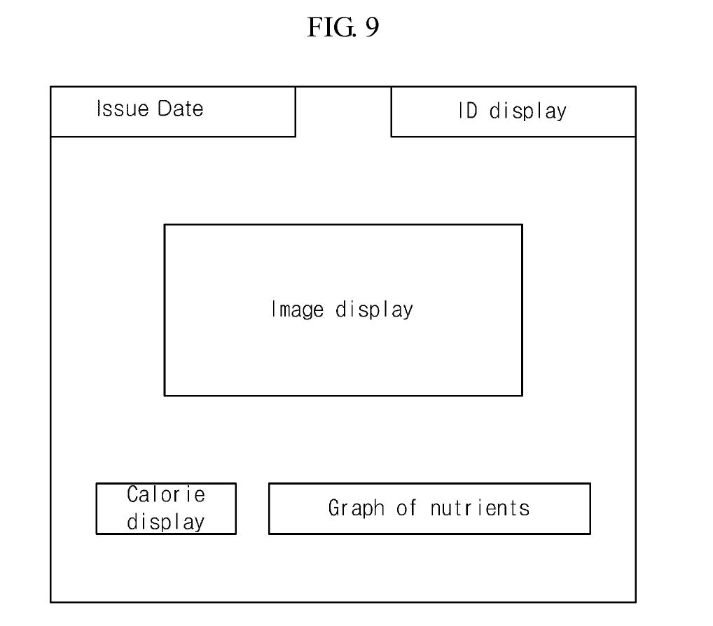

With reference to FIG. 9, the illustrated results report displays the I.D. and the issued date at the upper portion thereof; individual samples chosen by the user, and the loading modules, together with images, at the central portion thereof; and the nutrient composition and caloric content of the mixed sample by kind graphically at the bottom portion thereof.

The results report may contain a table of health functions for individual nutrients on one side. For example, health information, such as information that the mixed sample rich in dietary fiber is preventive of hypertension and effective for weight loss, may be displayed.

For reuse, the results report may be managed using bar codes.

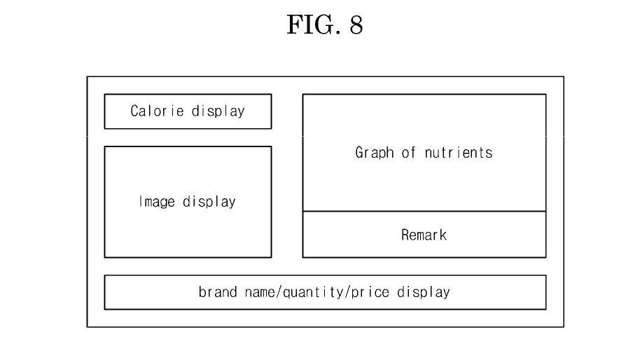

Referring to FIG. 8, shown is a display of the information extracted from the capsule and the loading module by the reader. As can be seen, a content similar to the results report is displayed by extracting information from a recipe to be mixed by the user from the capsules loaded into the loading modules while the price, predetermined according to the capsule and loading module selected by the user, is displayed, as well, to readily identify the total price.

Below, a description will be given of the health monitoring method of the present invention using the apparatus, with reference to the drawings.

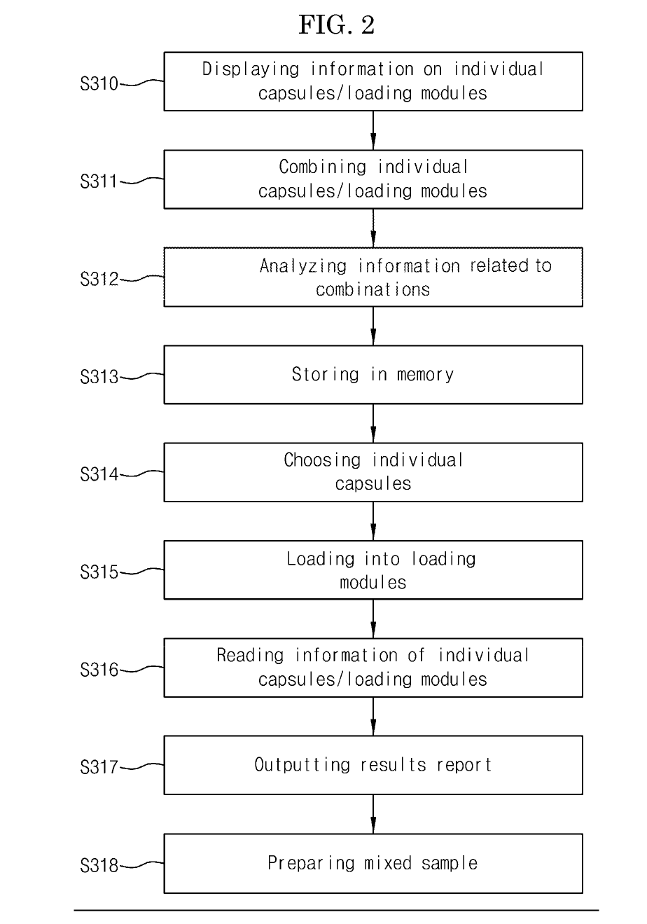

FIG. 2 is a flow chart illustrating the health monitoring method of the present invention. As illustrated, the health monitoring method of the present invention starts with classifying individual samples by weight and displaying the classified information on the individual capsules 300 (S310).

More specifically, information about at least one of: the kind, place of origin, shipment date, weight, price, image, and nutrient composition or caloric content of each sample by weight is displayed on individual capsules 300.

The kind and weight of each sample may be displayed on each side of the capsule. The capsules may be set to have different heights depending on their weights, and different colors depending on their kind, so that they can be readily discriminated from each other by weight and kind.

The loading modules, configured to accommodate a plurality of capsules 300, are provided by the kind of recipe, and combinations of the capsules with the loading modules 210 are set and analyzed (S311-S312).

More specifically, in consideration of cases where recipes and individual capsules might be mixed, the combinations are made, and analyzed.

After step (S311), information on the nutrient composition or caloric content to be taken from the mixed samples of individual capsules 300 in the loading modules 210 is stored in the memory of the control unit 110 (S313).

Steps (S310) to (S313) are set to prepare the capsules 300 and the loading modules 210, and to store relevant data in the memory 116.

After completion of the steps S310 to S313, the user chooses capsules 300 to be loaded into the loading modules 210. More specifically, the loads of individual capsules 300 depending on the kinds and weights of foods are loaded into the loading modules 210 in order (S315).

When all of the necessary capsules 300 are determined to be loaded into the loading modules 210 in step S315, the control unit 110 reads information from the information modules 121˜121n of the individual capsules 300, the recipe information from the loading modules 210, and information regarding a mixture of samples from individual capsules 300 loaded into the loading modules 210 from the read information of individual capsules 300 and loading modules 210 into the memory 116 to analyze the nutrient composition and caloric content of the mixed sample, and displays the analyzed information on a display device (S316).

In addition, the control unit 110 generates a results report with the information of the individual capsules 300 loaded into the loading modules 210 used as input sample information, and the analyzed information of the mixed sample serving as mixed sample information, to store the results report by I.D. in the memory 116, and to output the results report, along with the I.D. (S317).

The results report made in step S317 may be accessible by the user. If necessary, the results report may be read by a reader 112 set up in a counter, and the information on the mixed sample corresponding to the I.D. of the read results report is extracted from the memory 116, whereby the same mixed sample with the same nutrient composition or caloric content may be replicated (S318).

In this regard, the results report, as shown in FIG. 9, may display the I.D. and the issued date at the upper portion thereof; individual samples chosen by the user, and the loading modules, together with images, at the central portion thereof; and the nutrient composition and caloric content of the mixed sample by kind graphically at the bottom portion thereof.

In addition, the control unit 110 reads the I.D. of the results report, and displays information on the mixed sample corresponding to the read I.D. As needed, the control unit 110 may modify the displayed information of the mixed sample, and may extract the information on the mixed sample before and after the modification to allow for the preparation of a mixed sample, which is identical in nutrient composition and caloric content to the modified mixed sample.

Although the exemplary embodiments of the present invention have been disclosed for illustrative purposes, those skilled in the art will appreciate that various modifications, additions, and substitutions are possible, without departing from the scope and spirit of the invention as disclosed in the accompanying claims.

1. A health monitoring method for maintaining health, using a system comprising individual capsules including a first recording medium on which sample information about at least one of a kind, place of origin, shipment date, weight, price, image, and nutrient composition or caloric content by weight is recorded; a loading module including one or more receiving cells adapted to accommodate a plurality of the individual capsules, and a second recording medium on which recipe information about different beverages, price information about individual recipes, and images of the beverages are recorded; a memory of a control unit in which the recipe information of the loading module, and information about nutrient composition or caloric content taken from mixtures of samples from individual capsules designed to fit the loading module are stored, whereby data of the memory is managed; the health monitoring method comprising:
(a) classifying samples by weight and kind, and displaying the classified information on the individual capsules;
(b) accommodating at least one selected from among the individual capsules with the loading module;
(c) reading the information displayed on the individual capsules loaded on the loading module, and the recipe information of the loading module; and
(d) reading information on a mixed sample in which the recipe information on each individual sample, and the information of the loading module, both read in step (c), are mixed with each other, using the memory, and analyzing and displaying information on nutrient composition or caloric content of the mixed sample.

2. The health monitoring method of claim 1, wherein step (d) further comprises displaying at least one selected from among images of the loading module and the individual capsules designed to fit the loading module, recipe information of the loading module, the kinds of the individual capsules, quantities and prices of recipes and the individual capsules by kind, nutrient composition or caloric content of the mixed samples, and uptake efficient information extracted from the nutrients.

3. The health monitoring method, further comprising:
(e) allowing the control unit to generate a results report in which the recipe information of the loading module and the information of the individual capsules accommodated with the loading module of step (b) are used as input sample information while the analyzed information of the mixed sample of step (d) is used as a mixture sample information, storing the results report in the memory by identification (I.D.), and outputting the results report, together with the I.D.; and
(f) allowing the control unit to read the I.D. from the output results report and to extract the information of the mixed sample corresponding to the read I.D. from the memory, thereby preparing a mixed sample identical in nutrient composition or caloric content to the read mixed sample.

4. The health monitoring method of claim 3, wherein the results report of step (e) displays at least one selected from among images of the loading module and the individual capsules accommodated with the loading module, nutrient composition or caloric content of the mixed samples, and information regarding uptake efficiency extracted from the nutrients by I.D.

5. The health monitoring method of claim 3, wherein step (f) further comprises allowing the control unit:
(f-1) to read the I.D. of the results report and display information of the mixed sample corresponding to the read I.D.;
(f-2) to modify the displayed information of the mixed sample; and
(f-3) to extract the modified information of the mixed sample from the memory, thereby replicating a mixed sample identical in nutrient composition or caloric content to the modified mixed sample.

6. The health monitoring method of claim 1, wherein each of the receiving cells is provided with a holding step, and each of the capsules is provided with a groove at a location corresponding to the holding step of the receiving cell, wherein when the capsule is received in the loading module, the groove of the capsule is removably engaged with the holding step of the receiving cell.

7. The health monitoring method of claim 1, wherein each of the capsules is formed by a combination of one or more cylindrical bodies, in which each of the cylindrical bodies is provided with a female locking portion in an upper part thereof and a male locking portion in a lower part thereof, wherein the male locking portion of a cylindrical body is combined with the female locking portion of another cylindrical body.

8. The health monitoring method of claim 5, wherein the cylindrical bodies have one or more different colors, images, or patterns according to the kinds or weight of the samples.

9. The health monitoring method of claim 5, wherein the loading module is displayed with different colors, images, or patterns depending on the kind of recipe.

10. The health monitoring method of claim 4, wherein each of the capsules is provided with a vertical groove on a surface thereof, and the loading module is provided with a vertical protrusion on an inner surface thereon, wherein when the capsule is received in the loading module, the vertical groove of the capsule is movably engaged with the vertical protrusion of the loading module and one or more capsules can be loaded into the loading module.
